(19) United States
(12) Patent Application Publication
(10) Pub. No.: US 2008/0003641 A1
(43) Pub. Date: Jan. 3, 2008

(54) THE PRODUCTION OF HIGH-ACTIVITY SUPEROXIDE DISMUTASE (SOD) AND APPLICATION IN METHODS OF BOTH THE SOLID-STATE AND LIQUID-STATE FERMENTATION

(76) Inventors: Pao-Chuan Hsieh, Nei Pu Hsiang (TW); Chang-Yu Chuang, Nei Pu Hsiang (TW)

Correspondence Address: LEONG C LEI, PMB # 1008, 1867 YGNACIO VALLEY ROAD, WALNUT CREEK, CA, 94598, US

(21) Appl. No.: 11/383,496

(22) Filed: May 16, 2006

(51) Int. Cl. C12N 9/08; C12P 21/06

(52) U.S. Cl. 435/69.1; 435/192; 435/252.3

(57) ABSTRACT

The present invention of “A production method for high-activity superoxide dismutase (SOD) and application in methods of both the solid-state and liquid-state fermentation” lies in processing the strains of Bacillus subtilis with quantifying the activity of superoxide dismutase (SOD) in fermentation broth by nitro blue tetrazolium (NBT) reduction method, and then one kind of high-activity SOD-producing strain, which will be acquired while after the dialysis process, named Strain S-8 and afterward, this strain S-8 will be utilized to produce SOD by performing the liquid-state and solid-state fermentation methods respectively.

### BACKGROUND OF THE INVENTION

(a) Technical Field of the Invention

The present invention relates to the utilization of strain of Bacillus Subtilis, wherein the dialysis process is performed in order to acquire the high-activity SOD-producing strain by performing the liquid-state and solid-state fermentation methods respectively and, in addition, to study the optimum cultivation conditions for said strain during the production of SOD.

(b) Description of the Prior Art

Reactive Oxygen Species (ROS) comprises free radicals, such as Superoxide Anion Free Radicals (O2—.), Hydrogen Peroxide (H2O2) and Hydroxyl Free Radicals (HO.) etc., which are matters generated unavoidably by the living organisms during oxidative metabolism.

In recent years, the studies related to the Reactive Oxygen Species (ROS) has already been proved that it has a toxicity to cells and is applicable to activate the mutations of living organisms. The free radicals can oxidize unsaturated fatty acids on cell membrane and hence causing a peroxidation reaction, destroying cell membranes and, in addition, resulting in oxygenation of protein as well as damages to DNA. In order to prevent from the attacks by free radicals, thus the living organisms has developed a kind of defense mechanism, such as non-enzymatic and enzymatic defense mechanism, wherein the non-enzymatic defense mechanism comprising matters of iso-ascorbic acid and tocophenol etc. and the enzymatic defense mechanism comprising matters of superoxide dismutase (SOD; EC 1.15.1.1), and catalase etc. so as to eliminate free radicals inside the human body as well as relief the condition with excessive free radicals.

Superoxide dismutase (SOD) is a kind of protein having a metal matter positioned at the activity center, being classified into three classifications according to metal combined thereat; Cu/Zn Superoxide dismutase (Cu/Zn-SOD), Fe Superoxide dismutase (Fe-SOD) and Mn Superoxide dismutase (Mn-SOD), SOD is the earliest cleaned enzyme while generating the reactive oxygen species (ROS) inside the human body, where the relevant mechanism is to transform the superoxide anion fee radicals generating firstly in the reactive oxygen species (ROS) into hydrogen peroxide (H2O2) and oxygen molecule, by dismutation in order to relief the accumulation of excessive reactive oxygen species (ROS) inside the human body.

The current studies related to the SOD are mostly focusing on the reactive units inside the SOD itself, such, as the comparison of content of superoxide dismutase (SOD) among Taiwanese medicinal mushrooms, or producing SOD with the fungi—Aspergillus Oryzae by performing the solid-state fermentation method, so as to study the activity and yield, however due to the fermentation time of fungi is much longer than others, thus mere there is very few seldom to discuss and study in the field of creating SOD activity with repaid growing germ by the fermentation method.

Additionally, there are various applications of SOD currently, for example, SOD has already been used in the clinical medicine, e.g. in improving patient's autoimmune diseases, inflammatory disease treatments and relief of muscular fatigue, regarding to other applications, where the SOD is also applied in the developments of the functional foods, food fortification and dietary supplements as well as cosmetics, therefore, if we can find out the high-yield type SOD-producing strains among the microorganism, producing the uniform quality products in mass production with a low cost, thus enhancing the whole economic benefits while acting as an additive in industries of health foods, cosmetics and pharmaceutics.

### SUMMARY OF THE INVENTION

The present invention of “A production method for high-activity superoxide dismutase (SOD) and application in methods of both the solid-state and liquid-state fermentation” lies in providing the mass production of Bacillus Subtilis in order to acquire the high-activity SOD-producing strain, named Strain S-8, which has been deposited at the Food Industry Research and Development Institute located in Hsinchu City, Taiwan, on the date of Feb. 9, 2006 with the Depositary Serial No. BCRC 910306.

The present invention of “A production method for high-activity superoxide dismutase (SOD) and application in methods of both the solid-state and liquid-state fermentation” lies in performing the dialysis process in order to acquire the high-activity SOD-producing strain by performing the liquid-state and solid-state fermentation methods respectively and, in addition, to provide the optimum cultivation conditions for the said strain during the production of SOD.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

The present invention will be apparent to those skilled in the art by reading the following detailed description of a preferred embodiment thereof, with reference to the attached drawings, in which;

FIG. 1 illustrates variation related to SOD activity, pH value and total bacterial counts for the strain of Bacillus Subtilis S-8 during the process of liquid-state fermentation;

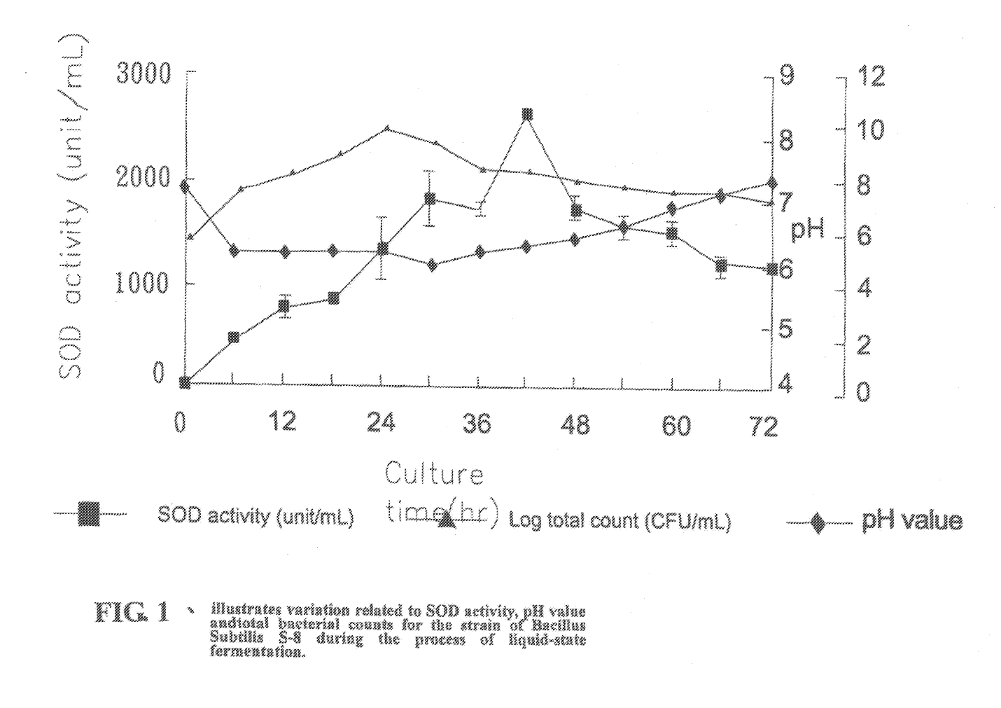

FIG. 2 illustrates variation related to SOD activity, pH value and total bacterial counts for the strain of Bacillus Subtilis S-8 during the process of solid-state fermentation.

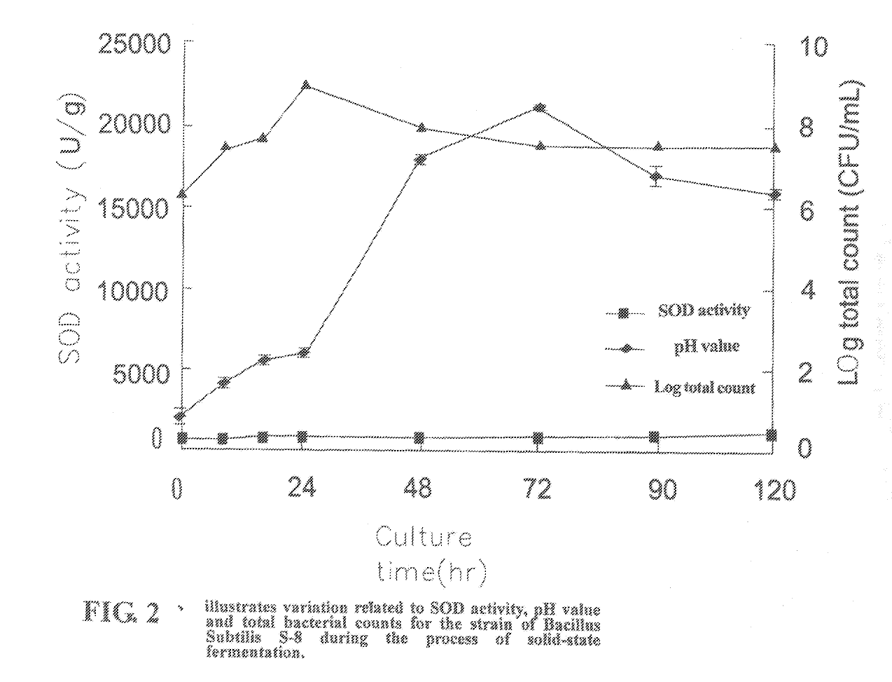

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

The present invention relates to a kind of production method for high-activity superoxide dismutase (SOD) and application in methods of both the solid-state and liquid-state fermentation lies in filtrating Natto product bought from the local markets to generate the 270 strains of Bacillus Subtilis and then quantifying the activity of superoxide dismutase (SOD) in fermentation broth by nitro blue tetrazolium (NBT) reduction method and, in addition, it is applicable to acquire the high-activity SOD-producing strain, while after the dialysis process, which, is named Strain S-8, and afterward this strain S-8 will be utilized to produce SOD by performing the liquid-state and solid-state fermentation methods respectively.

Without the original studying in nourishment condition, the Strain S-8 is capable to produce the SOD with an activity of 153 U/mL, and it is applicable to achieve the nourishment condition of optimum rapid growth by performing the liquid-state fermentation method, in which the groups or combinations for carbon source consisting of glucose, D-sorbitol solution, and dissoluble starch, and the groups or combinations for the nitrogen source consisting of soybean powder, beef extract, and peptone, with a pH value of about 6˜7.5, more over, the Strain S-8 can produce the superoxide dismutase (SOD) with an improved activity of 2600 U/mL.

The production capability of Strain S-8 is performed via an experiment by the solid-state fermentation method, wherein solid-state fermentation substrate comprising the combination of wheat acrospire in 10˜20 mesh diameter and soybean together with the additive of 5% sugar, such as the groups or combinations of glucose, D-sorbitol solution, dissoluble starch, in order to perform the solid-state fermentation process, for fermenting at least 72 hours, wherein the activity of superoxide dismutase (SOD) contained in fermentation product can reach up to 21,000 U/g, which is much higher than the control group with 645 U/g which didn't have any Strain S-8 at all.

Although the present invention has been described with reference to the preferred embodiment thereof, it is apparent to those skilled in the art that a variety of modifications and changes may be made without departing from the scope of the present invention which is intended to be defined by the appended claims.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A kind of high-activity superoxide dismutase (SOD), named Strain S-8, where the
said Strain S-8 has been deposited at the Food industry Research and Development Institute located in Hsinchu City, Taiwan, on the date of Feb. 9, 2006 with the Depositary Serial No. BCRC 910306.

2. A kind of production method for high-activity superoxide dismutase (SOD) and application in methods of both the solid-state and liquid-state fermentation lies in filtrating Natto product to generate the strains of Bacillus Subtilis and then quantifying the activity of superoxide dismutase (SOD) in fermentation broth by nitro blue tetrazolium (NBT) reduction method and, in addition, it is applicable to acquire the high-activity SOD-producing strain, while after the dialysis process, which is named Strain S-8, and afterward this strain S-8 will be utilized to produce SOD by performing the liquid-state and solid-state fermentation methods respectively.

3. The liquid-state fermentation method as claimed in claim 2, wherein it is applicable to achieve the nourishment condition of optimum rapid growth, in which the groups or combinations for carbon source consisting of glucose, D-sorbitol solution, and dissoluble starch etc., and the groups or combinations for the nitrogen source consisting of soybean powder, beef extract, and peptone etc.

4. The liquid-state fermentation method as claimed in claim 3, wherein its pH value is about 6˜7.5.

5. The solid-state fomentation method as claimed in claim 2, wherein it comprises the combination of wheat acrospire and soybean together with the additive of sugar in order to perform die solid-state fermentation process, for fermenting at least 72 hours.

6. The solid-state fermentation method as claimed in claim 5, wherein it comprises the combination of wheat acrospire and soybean in size of 10˜20 mesh diameter.

7. The solid-state fermentation method as claimed in claim 5, wherein the additive of sugar is the groups or combinations of such as glucose, D-sorbitol solution, and dissoluble starch, etc.

8. The solid-state fermentation method as claimed in claim 5, wherein the proportional ratio for sugar additive is 5%.
